(19) United States
(12) Patent Application Publication
(10) Pub. No.: US 2006/0182362 A1
(43) Pub. Date: Aug. 17, 2006

(54) Systems and methods relating to enhanced peripheral field motion detection

(76) Inventors: Peter McLain, Bellingham, WA (US); Rick Mancilla, Ventura, CA (US); Edward Steiner, Owings Mills, MD (US); Andrew Haring, Kirkland, WA (US)

Correspondence Address: GRAYBEAL, JACKSON, HALEY LLP, 155 - 108TH AVENUE NE, SUITE 350, BELLEVUE, WA, 98004-5901, US

(21) Appl. No.: 11/286,135

(22) Filed: Nov. 23, 2005

Related U.S. Application Data
(63) Continuation-in-part of application No. 11/165,824, filed on Jun. 23, 2005.
(60) Provisional application No. 60/630,824, filed on Nov. 23, 2004.
(60) Provisional application No. 60/665,967, filed on Mar. 28, 2005.

(51) Int. Cl. G06K 9/40; A61B 5/05

(52) U.S. Cl. 382/254; 600/407

(57) ABSTRACT

Systems and methods, etc., and the like for harnessing the human peripheral field motion detection (“PFMD”) system to detect and analyze features in a digital image, including the interpretation of images such as radiographic images in medical and other fields. The systems, methods, etc., include providing a series of images then providing and indicator such as applying magnitude enhancement analysis to an image selected due to recognition of the image by the PFMD by pausing at the image.

### CROSS-REFERENCE TO RELATED APPLICATIONS

The present application claims priority from U.S. provisional patent application No. 60/630,824 filed Nov. 23, 2004; U.S. provisional patent application No. 60/665,967 filed Mar. 28, 2005; and, U.S. patent application Ser. No. 11/165,824, filed Jun. 23, 2005, which are incorporated herein by reference in their entirety and for all their teachings and disclosures.

### BACKGROUND

To date, most automated image interpretation systems have centered on enhancing the images to improve or ease detection by the human central visual system. For example, systems to interpret X-ray images, MRI images, CAT scans, etc., have provided a variety of approaches to increase lesion conspicuity to the human central visual system.

Examples of such systems include the Z-axis kinematic (ZAK) systems sometimes known as magnitude enhancement analyses provided by LumenIQ and discussed in several patents and patent applications including U.S. Pat. No. 6,445,820; U.S. Pat. No. 6,654,490; US 20020114508; WO 02/17232; 20020176619; 20040096098; 20040109; 20050123175; U.S. patent application Ser. No. 11/165,824, filed Jun. 23, 2005; and, U.S. patent application Ser. No. 11/212,485, filed Aug. 26, 2005. Generally, these methods and systems use 3D visualization to improve a person's ability to see small differences in at least one desired characteristic in an image, such as small differences in the lightness or darkness (grayscale data) of a particular spot in a digital image using magnitude enhancement analysis. For example, these systems can display grayscale (or other desired intensity, etc.) data of a 2D digital image as a 3D topographic map: The relative darkness and lightness of the spots (pixels) in the image are determined, then the darker areas are shown as “mountains,” while lighter areas are shown as “valleys” (or vice-versa). In other words, at each pixel point in an image, grayscale values are measured, projected as a surface height (or z axis), and connected through image processing techniques. The magnitude enhancement analysis can be a dynamic magnitude enhancement analysis, which can comprise at least one of rolling, tilting or panning the image, which are examples of a cine loop. FIGS. 1A and 1B show examples of this, where the relative darkness of the ink of two handwriting samples are shown in 3d with the darker areas shown as higher “mountains.” These techniques can be used with any desired image, such as handwriting samples, fingerprints, DNA patterns (“smears”), medical images such as MRIs, x-rays, industrial images, satellite images, etc.

Another well-known human visual system is the peripheral field motion detection (“PFMD”) system (also known as the peripheral motion detection system), Levi et al., Vision Res. Vol.24. No.8, pp. 789-800, 1984, which has substantial sensitivity and could be useful in interpreting radiographic images. Generally speaking, the PFMD system detects motion detected in the periphery of a person's vision. Much of the sensitivity in PFMD may be based upon activation of primal pathways in human optic sensory systems. For example, using only the central vision system, we often fail to see a still bird camouflaged among tree leaves; when we stare intently at a still object, it can be difficult to detect even when we “know what we're looking for”—and all the more difficult when we don't. Movement of the bird wings, even subtle movement, will often activate PFMD. Once the observer detects the bird with his or her PFMD, he/she can then track and immediately focus on it using his or her central vision system. In vision science, this may be referred to as a “hand off between the PFMD system and the central vision system: PFMD first detects the object, and the central vision system then focuses on the same object to determine its detailed characteristics.

There has gone unmet a need for improved systems and methods, etc., for interpreting the analysis of images, such as medical images, using PFMD system. The present systems, methods, etc., provide these or other advantages.

### SUMMARY

The present discussion includes systems, apparatus, technology and methods and the like for harnessing the PFMD system to detect and analyze features in a digital image, including the interpretation of images such as radiographic images in medical and other fields.

In one aspect, the systems, methods, etc., herein include comprise identifying at least one image from a series of related images to subject the image to further analysis. This can comprise: a)scrolling through a series images, each image having at least 2-dimensions, at a frame rate adequate for changes from one image to the next to invoke the viewer's peripheral field motion detection (“PFMD”) system upon determination of apparent motion upon transition from one image to the next in the series; b)automatically determining when the viewer pauses at a given image; c)automatically stopping the series of image at the given image in response to the pause; and d)providing an indicator indicating that viewer paused at the given image. In certain embodiments, the methods, etc., further comprise subjecting the given image to magnitude enhancement analysis to provide a magnitude enhanced image, such that at least one relative magnitude across at least a substantial portion of the image can be depicted in an additional dimension relative to the at least 2-dimensions such that additional levels of at least one desired characteristic in the image can be substantially more cognizable to the viewer's eye compared to the 2-dimensional image without the magnitude enhancement analysis.

The frame rate can be controlled by the viewer, the length of the pause adequate to invoke the stop can be automatically determined or set by the user, and typically the pause must last longer than an automatically predetermined amount of time, for example more than about 0.05, 0.1, 0.2, 0.3, 0.5, or 1.0 seconds. The image can be a digital conversion of a photographic image and the magnitude enhanced image can be displayed to the viewer as a cine loop, which can comprise an automatically determined animation of at least one of roll, tilt or pan, or can be determined by the user, who can, for example, vary at least one of the roll, tilt, pan, angle and apparent location of the light source in the cine loop. In other words, the user can set the cine loop or vary features or aspects of a cine loop that has been automatically set. The cine loop can be rotated in an about 30-60 degree arc or other arc as desired, such as 10°, 20°, 40°, 45°, 50°, or 70°.

The ZAK analysis comprises an enhanced magnitude in a further dimension (e.g., showing grayscale in a third, z dimension relative to the x,y dimensions if a typical 2-D image. The magnitude can also or instead comprise at least one of hue, lightness, or saturation, or a combination of values derived from at least one of grayscale, hue, lightness, or saturation. The magnitude can also comprise or be an average intensity defined by an area operator centered on a pixel within the image, and can be determined using a linear or non-linear function.

The series of images can be medical radiographic images such as MRI, CAT, X-ray, MRA, and vascular CTA. The series of images can also be forensic images, from an industrial manufacturing plant, satellite photographic images, fingerprint, palmprint or footprint images and/or non-destructive examination images.

The series of images can comprise a laterally-moving series of images whereby a subject can be sliced by the images, can comprise a series of images recorded of substantially the same site and over time, and/or can comprise a video or movie image sequence. The methods, etc., can further comprise automating at least one image variable selected from the group consisting of Z-axis height, roll, contrast, center movement, and directional lighting, which lighting can comprise holding the image stationary, and alternating the apparent lighting of an object in the image between point source lighting and directional lighting, and/or moving the light source between different positions in the image. The image variable can also be apparent motion within the image such as where the center of an object in the image appears to move closer to the screen, then recedes from it. The methods, etc., further comprise providing at least one of a sound indicator or an optical indicator that indicates the pause occurred.

In further aspects, the methods, systems, etc., can comprise computer-implemented programming that performs the automated elements discussed herein, and a computer comprising such computer-implemented programming. The computer can comprise a distributed network of linked computers, handheld computer, a wirelessly connected computer. The computer can also comprise networked computer system comprising computer-implemented programming that performs the automated elements, which can be implemented on the handheld wireless computer.

These and other aspects, features and embodiments are set forth within this application, including the following Detailed Description and attached drawings. Unless expressly stated otherwise or clear from the context, all embodiments, aspects, features, etc., can be mixed and matched, combined and permuted in any desired manner. In addition, various references are set forth herein, including in the Cross-Reference To Related Applications, that discuss certain systems, apparatus, methods and other information; all such references are incorporated herein by reference in their entirety and for all their teachings and disclosures, regardless of where the references may appear in this application.

### BRIEF DESCRIPTION OF THE DRAWINGS

FIGS. 1A and 1B show examples of magnitude enhancement analysis processing of two handwriting samples with the darker areas shown as higher “mountains.”

FIG. 2 shows an initial user interface of an embodiment of a Smart Activation Module (“SAM”) configuration.

FIG. 3 shows another variation of the interface displaying eight series of images.

FIG. 4 shows the SAM after it has been activated by the user's pause on a particular image in a series.

FIG. 5 shows the same image as FIG. 4, at the other end of the interrogation sweep arc.

FIG. 6 shows an alternative view of the same image as FIG. 4.

FIG. 7 shows another alternative starting screen, depiction of the images in “tile mode”, where all images in a series are shown simultaneously.

FIG. 8 shows tile mode with window leveling enhancement activated in the top tile of column 2.

FIG. 9 shows the leveling enhancement of FIG. 8 applied to the other images in the series.

### DETAILED DESCRIPTION

In certain embodiments, the present systems, methods, etc., provide systems and approaches to display and analyze images using the human PFMD system and automated invocation of an indicator of the invocation of the PFMD such as automated application of magnitude enhancement analysis, usually on the basis of the person examining a series of images pausing on an image that “caught the attention” of the person's PFMD.

Turning to an exemplary embodiment, in the field of medical radiology, a radiologist typically sits at his or her computer workstation reviewing a series of images, with each image representing a 2D “slice” through the target anatomical structure. In a typical examination workflow, the radiologist scrolls quickly through an image set, and stops at particular 2D slice. The frame rate of the images as they pass by while scrolling is usually controlled by the human viewer but in certain embodiments the frame can be automatically controlled, in which case the invocation of PFMD is typically automatically determined by sensing indications from the human analyst other than pausing, for example by using an eye motion detection device.

When the radiologist pauses on a particular 2D slice for a pre-determined length of time, that image is automatically rendered in ZAK software to provide magnitude enhancement analysis. The predetermined length of time can be automatically determined, for example based on automatically sensed and reviewed viewing patterns either of people in general or the particular radiologist. The length of time can also be set manually by the user, or other person if desired.

The resulting 3D image is then presented on the system monitor with cursor controls and can be provided with an interrogation sweep arc (also called cine loop) that is automatically generated. For example, the sweep arc can roll the image back and forth so that variations in the 3D surface are easier to see. The sweep arc can be manually or automatically set and can be of a single length or variable. The viewing angle and/or angle at which the target image is represented of the sweep arc and other ZAK-rendered images can be pre-determined but can also be adjusted.

The combination of ZAK visualization and consistent motion pattern (typically both generated automatically) is designed to work in concert with PFMD. Once the radiologist detects a characteristic of potential interest through use of PFMD, his/her central visual system can then focus on the image/item of interest and perform a detailed analysis to determine clinical relevancy.

The workstation software and configuration discussed above can be referred to as a Smart Activation Module (“SAM”). Of course, any computer configuration can be used, including stand-alone personal computers, mainframes, handhelds, distributed networks, etc. SAM allows use of the PFMD to trigger central vision system analysis in a “real time” dynamic manner. Generally speaking, the SAM detects a pause, interprets it as “subliminal interest” and then activates an indicator that informs the analyst that the pause has been detected. The indicator can be as simple as a chime sound or flashing light, but typically includes one or more of the following features that are activated upon detection of pause/invocation of the viewer's PFMD. The SAM typically includes one or more of the following features:
(a) incorporation of motion (e.g., interrogation sweep arc) into a static image browser;
(b) automated activation of sweep arc when the analyst pauses for a pre-determined length of time (e.g., more than 0.05″, 0.1″, 0.2″, 0.3″, 0.5″, 1.0″); and
(c) automated initiation of 3D grayscale visualization and/or other ZAK technology features not already in use to increase conspicuity of image features when combined with motion (ZAK technology features can also be implemented into the viewing stream of the series of images during “motion”, if desired).

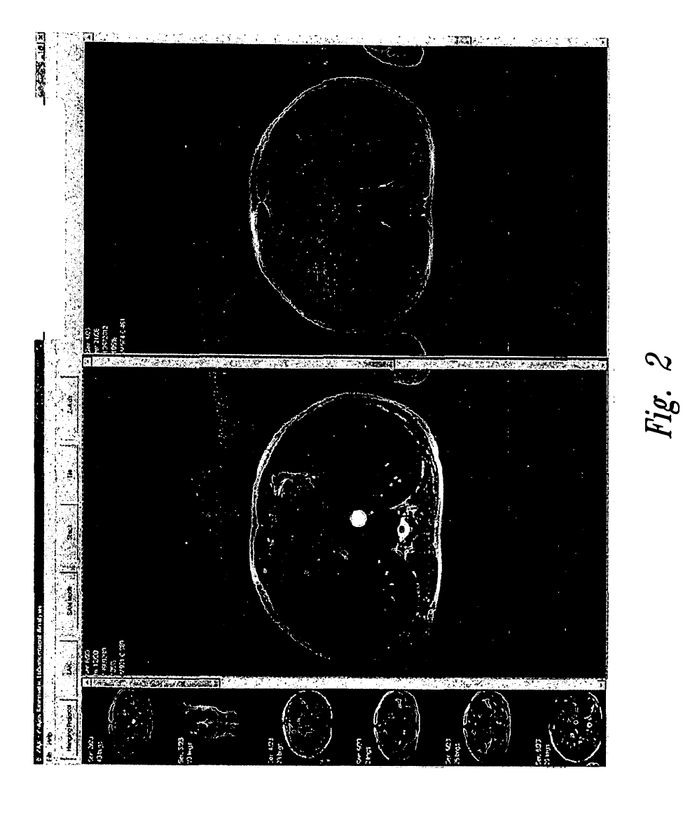

FIGS. 2-9 show one embodiment.

a. FIG. 2 shows an initial user interface of an embodiment of a SAM configuration. This particular screen shows a “study view” displaying two series of MRI images. The tiles at the far left side of the image show the various series available to the radiologist for more in-depth analysis.

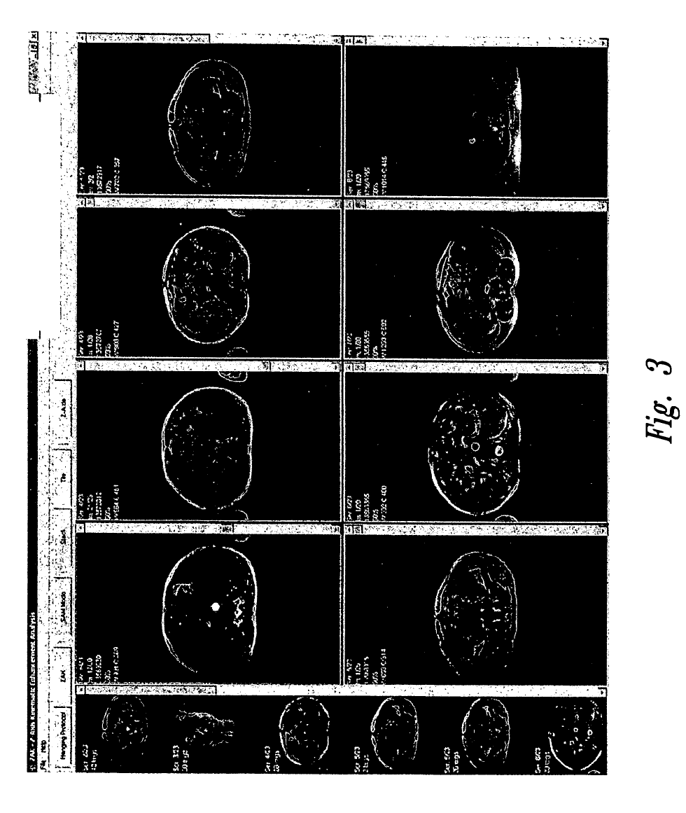

b. FIG. 3 shows another variation of the interface displaying a series of eight images.

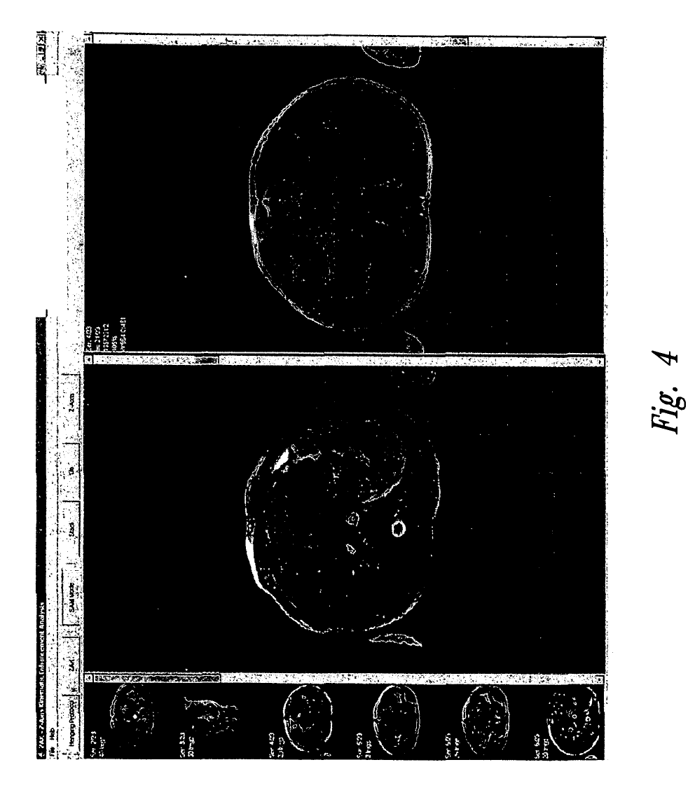

c. FIG. 4 shows the SAM after it has been activated by the user's pause on a particular image in the series. The 2D image file automatically renders the image to show grayscale variation (ZAK) in 3D, and the image then rotates in a pre-determined sweep arc.

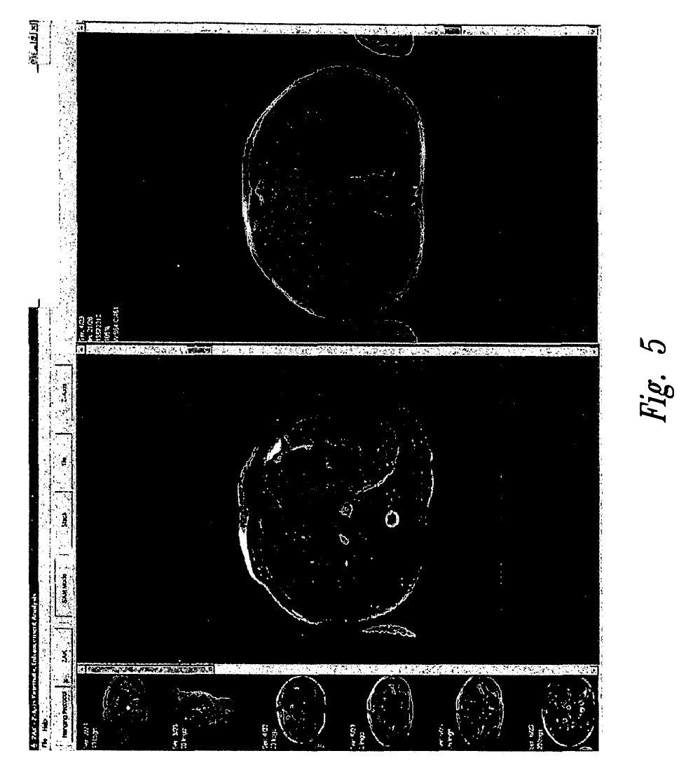

d. FIG. 5 shows the same image at the other end of the interrogation sweep arc.

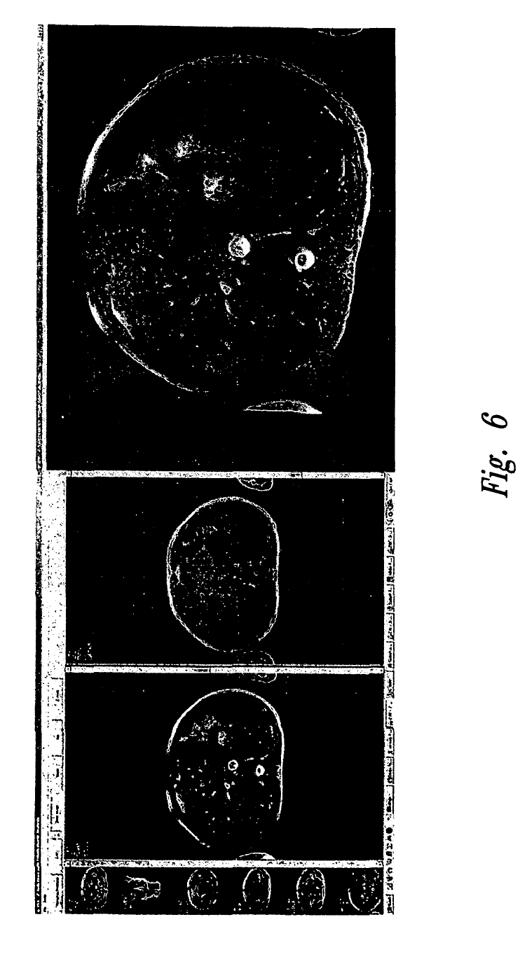

e. FIG. 6 shows an alternative view of the image. Here, the screen is configured so that the 3D, moving image is displayed in a separate window, and can be moved to a separate screen.

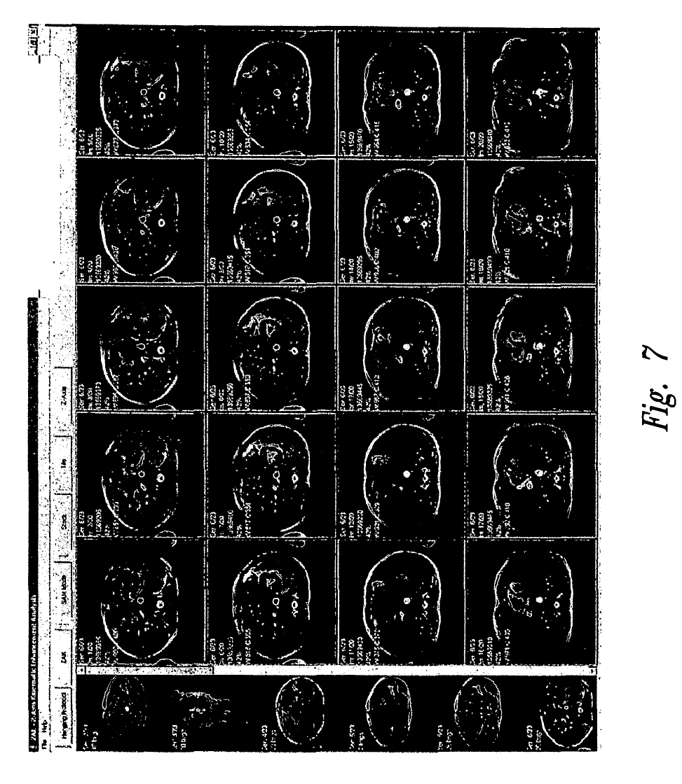

f. FIG. 7 shows another alternative starting screen: depiction of the images in “tile mode”, where all images in a series are shown simultaneously.

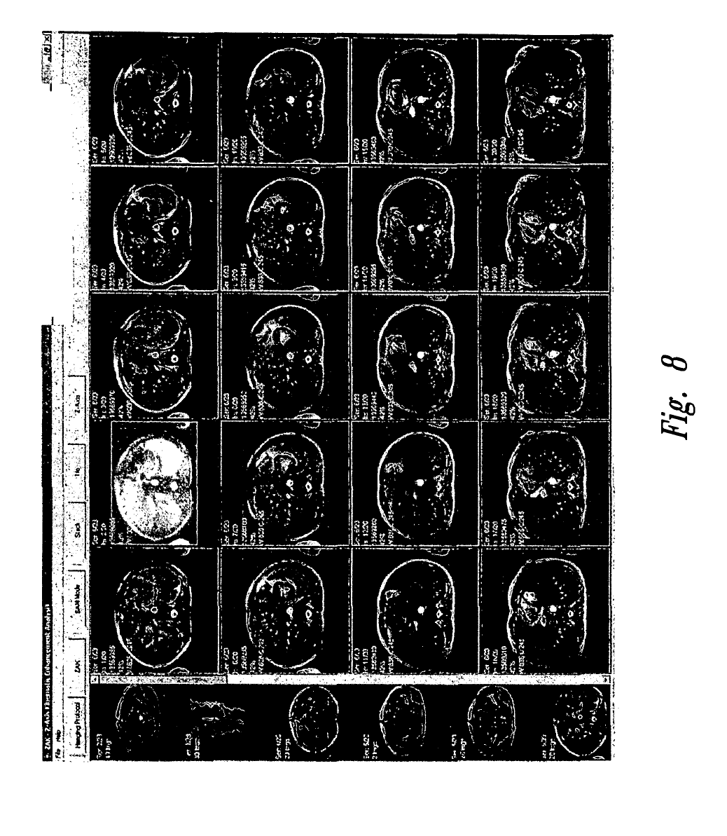

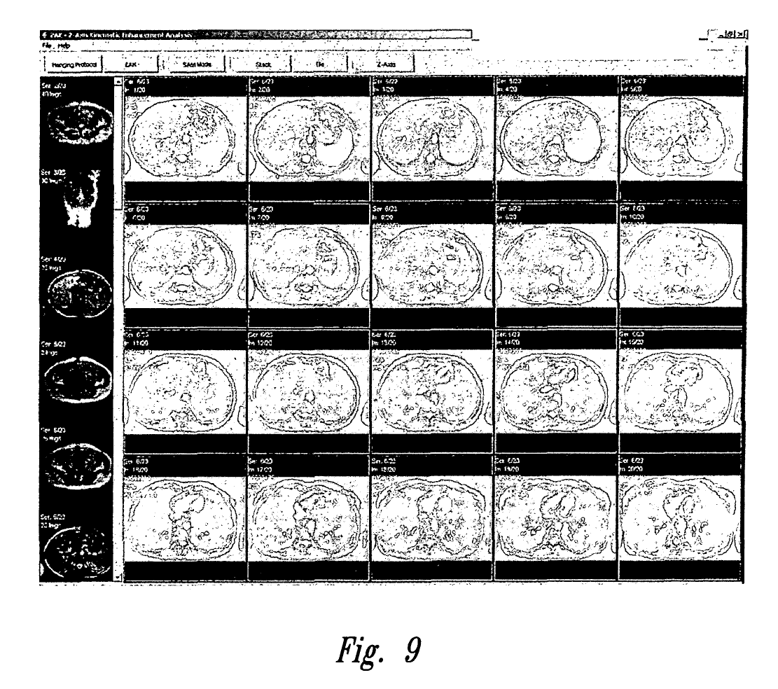

g. FIG. 8 shows tile mode with window leveling enhancement activated in the top tile of column 2. Window leveling settings are determined on a single image and are then automatically applied to all images in the series. FIG. 9 shows the leveling enhancement applied to the other images in the series.

The systems, methods, etc., also has additional applications in other domains where decisions are made based on human interpretation of images. For example, in the forensic domain, SAM can be used when a fingerprint, palmprint or footprint examiner is analyzing a series of print images to determine which one is a match to the latent fingerprint he/she is investigating, for example when using the AFIS system. When the examiner pauses on a selected print for a pre-determined amount of time, the print is automatically rendered in 3D and rotated at a 30-60 degree arc (cine loop). Similarly, a portion of the fingerprint could be selected for exposition in SAM.

Further embodiments apply to the field of Non-Destructive Examination (NDE). For example, industrial technicians review large numbers of x-rays of pipeline welds to determine whether any weld defects are present. The same visual principles apply: PFMD, triggered by 3D grayscale (or other suitable cue) visualization and motion, can identify potential defects that the central vision system then focuses on.

Alternative embodiments include

a. Different types of domain-specific images: The present systems, methods, etc., including SAM, can be used to analyze a variety of medical images besides CAT scan studies. Additional examples of multi-image sets include MRI, MRA, and Vascular CTA. For example, the images can be collected in a laterally-moving series approach (similar to a slicing loaf of bread) where a subject is “sliced” by the images, or the images can be of the same situs and recorded over time, in which case changes over time can appear as items in the field of view shrink, enlarge, are added or replaced, etc. Other combinations of images can also be used, such as video or movie image sequences. The combination of motion and ZAK visualization is also useful with single x-rays, such as lung images. Additional forensic images include palmprints, questioned documents, and ballistics. NDE images include metal plate corrosion, various weld types, and underground storage tanks. Any other desired image series can also be used, for example review of serial satellite photographs.

b. Types of motion: In one embodiment, SAM automatically pans and/or tilts the image in a back and forth motion, at a pre-determined interrogation sweep arc. SAM can also automate other image variables, such as Z-axis height, roll, contrast, center movement, and directional lighting, or any combination thereof. Two of these additional examples are discussed below:
i. Directional lighting: the image remains stationary, and the lighting alternates between point source lighting and directional lighting. Alternatively, the location of the light source can move between different positions in the image. These produce the effect of, among other things, turning on and off virtual “shadows” in a 3D image, which may highlight relevant features that are otherwise difficult to distinguish.
ii. Center movement: The center of the object moves closer to the screen, then recedes from it, usually in a regular pattern such as a regular “up and back” motion.

c. Types of image components visualized in 3D: A number of image features in addition to or instead of grayscale can be visualized in 3D. A further discussion of these other features can be found below and in some of the LumenlQ patents and patent applications cited herein. For example, SAM can provide the radiologist with a 3D visualization of hue, saturation, and a number of additional image components—whatever the examiner determines is relevant.

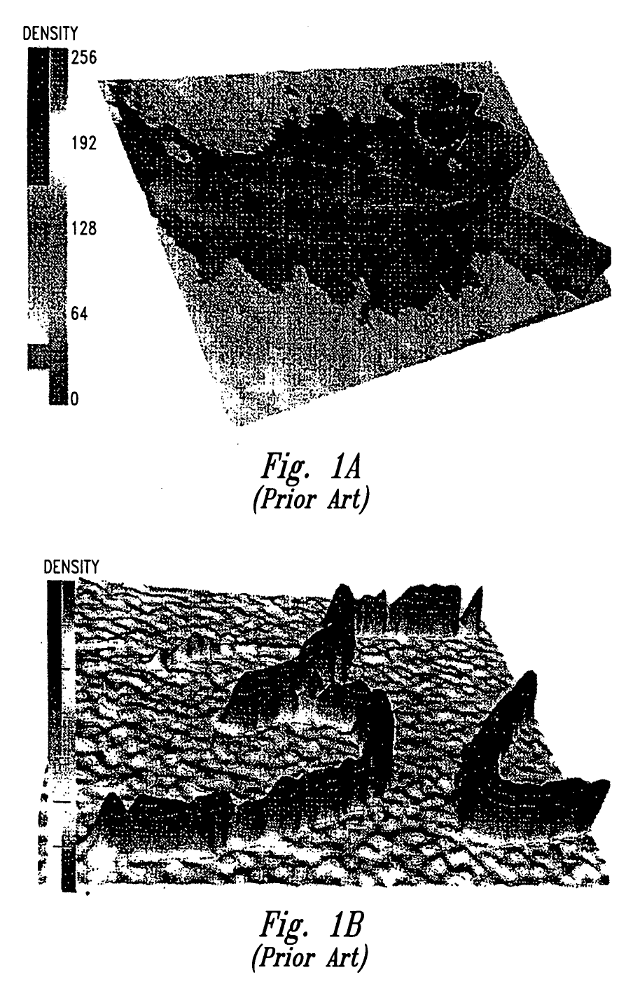

Turning to some general discussion of magnitude enhancement analysis/Z-axis kinematic (ZAK) systems, virtually any dimension, or weighted combination of dimensions in an at least 2D digital image (e.g., a direct digital image, a scanned photograph, a screen capture from a video or other moving image) can be represented as at least a 3D surface map (i.e., the dimension or intensity of a pixel (or magnitude as determined by some other mathematical representation or correlation of a pixel, such as an average of a pixel's intensity and its surrounding pixel's intensities, or an average of just the surrounding pixels) can be represented as at least one additional dimension; an x,y image can be used to generate an x,y,z surface where the z axis defines the magnitude chosen to generate the z-axis). For example, the magnitude can be grayscale or a given color channel. An example of a magnitude enhancement analysis based on grayscale is shown in FIGS. 1A and 1B. Various embodiments of ZAK can be found in U.S. Pat. No. 6,445,820; U.S. Pat. No. 6,654,490; US 20020114508; WO02/17232; US 20020176619; US 20040096098; US 20040109; US 20050123175; U.S. patent application Ser. No. 11/165,824, filed Jun. 23, 2005; and, U.S. patent application Ser. No. 11/212,485, filed Aug. 26, 2005.

Other examples include conversion of the default color space for an image into the HLS (hue, lightness, saturation) color space and then selecting the saturation or hue, or lightness dimensions as the magnitude. Converting to an RGB color space allows selection of color channels (red channel, green channel, blue channel, etc.). The selection can also be of single wavelengths or wavelength bands, or of a plurality of wavelengths or wavelength bands, which wavelengths may or may not be adjacent to each other. For example, selecting and/or deselecting certain wavelength bands can permit detection of fluorescence in an image, or detect the relative oxygen content of hemoglobin in an image. The magnitude can be determined using, e.g., linear or non-linear algorithms, or other mathematical functions as desired. The selection can also be of single wavelengths or wavelengths bands, or of a plurality of wavelengths or wavelength bands, which wavelengths may or may not be adjacent to each other. For example, selecting and/or deselecting certain wavelength bands can permit detection of fluorescence in an image, detect the relative oxygen content of hemoglobin in an image, or breast density in mammography.

Thus, the height of each pixel on the surface may, for example, be calculated from a combination of color space dimensions (channels) with some weighting factor (e.g., 0.5*red+0.25* green+0.25*blue), or even combinations of dimensions from different color spaces simultaneously (e.g., the multiplication of the pixel's intensity (from the HSI color space) with its luminance (from a YUV, YCbCr, Yxy, LAB, etc., color space)).

The pixel-by-pixel surface projections are in certain embodiments connected through image processing techniques to create a continuous surface map. The image processing techniques used to connect the projections and create a surface include mapping 2D pixels to grid points on a 3D mesh (e.g., triangular or rectilinear), setting the z-axis value of the grid point to the appropriate value (elevating based on the selected metric, e.g., intensity, red channel, etc.), filling the mesh with standard 3D shading techniques (gouraud, flat, etc.) and then lighting the 3D scene with ambient and directional lighting. These techniques can be implemented for such embodiments using modifications in certain 3D surface creation/visualization software, discussed for example in U.S. Pat. Nos. 6,445,820 and 6,654,490; U.S. patent application No. 20020114508; 20020176619; 20040096098; 20040109608; and PCT patent publication No. WO 02/17232.

The present invention can display 3D topographic maps or other 3D displays of color space dimensions in images that are 1 bit or higher. For example, variations in hue in a 12 bit image can be represented as a 3D surface with 4,096 variations in surface height.

Other examples of magnitude and/or display option include, outside of color space dimensions, the height of a gridpoint on the z axis can be calculated using any function of the 2D data set. A function to change information from the 2D data set to a z height takes the form f(x, y, image)=z. All of the color space dimensions are of this form, but there can be other values as well. For example, a function can be created in software that maps z height based on (i) a lookup table to a Hounsfield unit (f(pixelValue)=Hounsfield value), (ii) just on the 2D coordinates (e.g., f(x,y)=2x+y), (iii) any other field variable that may be stored external to the image, or (iv) area operators in a 2D image, such as Gaussian blur values, or Sobel edge detector values.

In all cases, the external function or dataset is related in some meaningful way to the image. The software herein can contain a function g that maps a pixel in the 2D image to some other external variable (for example, Hounsfield units) and that value is then used as the value for the z height (with optional adjustment). The end result is a 3D topographic map of the Hounsfield units contained in the 2D image; the 3D map would be projected on the 2D image itself.

Thus, the magnitude can be, for example, at least one or more of grayscale, hue, lightness, or saturation, or the magnitude can comprise a combination of magnitudes derived from at least one of grayscale, hue, lightness, or saturation, an average defined by an area operator centered on a pixel within the image. The magnitude can be determined using a linear or non-linear function.

As noted above, the processes transform the 2D grayscale tonal image to 3D by “elevating” (or depressing, or otherwise “moving”) each desired pixel of the image to a level proportional to the grayscale tonal value of that pixel in its' 2D form. The pixel elevations can be correlated 1:1 corresponding to the grayscale variation, or the elevations can be modified to correlate 10:1, 5:1, 2:1, 1:2, 1:5, 1:10, 1:20 or otherwise as desired. (As noted elsewhere herein, the methods can also be applied to image features other than grayscale, such as hue and saturation; the methods, etc., herein are discussed regarding grayscale for convenience.) The ratios can also be varying such that given levels of darkness or lightness have one ratio while others have other ratios, or can otherwise be varied as desired to enhance the interpretation of the images in question. Where the ratio is known, measurement of grayscale intensity values on a spatial scale (linear, logarithmic, etc.) becomes readily practical using conventional spatial measurement methods, such as distance scales or rulers.

The pixel elevations are typically connected by a surface composed of an array of small triangular shapes (or other desired geometrical or other shapes) interconnecting the pixel elevation values. The edges of each triangle abut the edges of adjacent triangles, the whole of which takes on the appearance of a surface with elevation variations. In this manner the grayscale intensity of the original image resembles a topographic map of terrain, where higher (mountainous) elevations could represent high image intensity, or density values. Similarly, the lower elevations (canyon-lands) could represent the low image intensity or density values. The use of a Z-axis dimension allows that Z-axis dimension to be scaled to the number of grayscale shades inherently present in the image data. This method allows an unlimited number of scale divisions to be applied to the Z-axis of the 3D surface, exceeding the typical 256 divisions (gray shades) present in most conventional images. High bit level, high grayscale resolution, high dynamic range image intensity values can, for example, be mapped onto the 3D surface using scales with 8 bit (256 shades), 9 bit (512 shades), 10 bit (1,024 shades) and higher (e.g., 16 bit, 65,536 shades).

As a surface map, the image representation can utilize aids to discrimination of elevation values, such as isopleths (topographic contour lines), pseudo-colors assigned to elevation values, increasing/decreasing elevation proportionality to horizontal dimensions (stretching), fill and drain effects (visible/invisible) to explore topographic forms, and more.

Turning to another aspect, digital images have an associated color space that defines how the encoded values for each pixel are to be visually interpreted. Common color spaces are RGB, which stands for the standard red, green and blue channels for some color images and HSI, which stands for hue, saturation, intensity for other color images. There are also many other color spaces (e.g., YUV, YCbCr, Yxy, LAB, etc.) that can be represented in a color image. Color spaces can be converted from one to another; if digital image pixels are encoded in RGB, there are standard lossless algorithms to convert the encoding format from RGB to HSI.

The values of pixels measured along a single dimension or selected dimensions of the image color space to generate a surface map that correlates pixel value to surface height can be applied to color space dimensions beyond image intensity. For example, the methods and systems herein, including software, can measure the red dimension (or channel) in an RGB color space, on a pixel-by-pixel basis, and generate a surface map that projects the relative values of the pixels. In another example, the present innovation can measure image hue at each pixel point, and project the values as a surface height.

The pixel-by-pixel surface projections can be connected through image processing techniques (such as the ones discussed above for grayscale visualization technology) to create a continuous surface map. The image processing techniques used to connect the projections and create a surface include mapping 2D pixels to grid points on a 3D mesh (e.g., triangular or rectilinear), setting the z axis value of the grid point to the appropriate value (elevating based on the selected metric, e.g., intensity, red channel, etc.), filling the mesh with standard 3D shading techniques (gouraud, flat, etc) and then lighting the 3D scene with ambient and directional lighting. These techniques can be implemented for such embodiments using modifications in Lumen's grayscale visualization software, as discussed in certain of the patents, publications and applications cited above.

From the foregoing, it will be appreciated that, although specific embodiments have been discussed herein for purposes of illustration, various modifications may be made without deviating from the spirit and scope of the discussion herein. Accordingly, the systems and methods, etc., include such modifications as well as all permutations and combinations of the subject matter set forth herein and are not limited except as by the appended claims.

1. A method of identifying at least one image from a series of related images to subject the image to further analysis, comprising:
a) scrolling through a series images, each image having at least 2-dimensions, at a frame rate adequate for changes from one image to the next to invoke the viewer's peripheral field motion detection (“PFMD”) system upon determination of apparent motion upon transition from one image to the next in the series;
b) automatically determining when the viewer pauses at a given image;
c) automatically stopping the series of image at the given image in response to the pause; and
d) providing an indicator indicating that viewer paused at the given image.

2. The method of claim 1 wherein the method further comprises subjecting the given image to magnitude enhancement analysis to provide a magnitude enhanced image, such that at least one relative magnitude across at least a substantial portion of the image is depicted in an additional dimension relative to the at least 2-dimensions such that additional levels of at least one desired characteristic in the image is substantially more cognizable to the viewer's eye compared to the 2-dimensional image without the magnitude enhancement analysis.

3. The method of claim 1 wherein the frame rate is controlled by the viewer.

4. The method of claim 1 wherein a length of the pause adequate to invoke the stop is automatically determined.

5. The method of claim 1 wherein a length of the pause adequate to invoke the stop is set by the user.

6. The method of claims 1 wherein the pause must last longer than an automatically predetermined amount of time.

7. The method of claim 6 wherein a length of the pause is more than about 0.3 seconds.

8. The method of claim 1 wherein the image is a digital conversion of a photographic image.

9. The method of claim 1 wherein the magnitude enhanced image is displayed to the viewer as a cine loop.

10. The method of claim 9 wherein the cine loop comprises an automatically determined animation of at least one of roll, tilt or pan.

11. The method of claim 9 wherein the cine loop is determined by the user.

12. The method of claim 11 wherein the user can vary at least one of the roll, tilt, pan, angle and apparent location of the light source in the cine loop.

13. The method of claims 1 wherein the magnitude is grayscale.

14. The method of claim 1 wherein the magnitude comprises at least one of hue, lightness, or saturation.

15. The method of claim 1 wherein the magnitude comprises a combination of values derived from at least one of grayscale, hue, lightness, or saturation.

16. The method of claim 1 wherein the magnitude comprises an average intensity defined by an area operator centered on a pixel within the image.

17. The method of claim 1 wherein the magnitude is determined using a linear function.

18. The method of claim 1 wherein the magnitude is determined using a non-linear function.

19. The method of claim 1 wherein the series of images are medical radiographic images.

20. The method of claim 19 wherein the medical radiographic images are at least one of MRI, CAT, X-ray, MRA, and vascular CTA.

21. The method of claim 1 wherein the series of images are forensic images.

22. The method of claim 1 wherein the series of images are images from an industrial manufacturing plant.

23. The method of claim 1 wherein the series of images are satellite photographic images.

24. The method of claim 1 wherein the series of images are fingerprint, palmprint or footprint images.

25. The method of claim 1 wherein the series of images are non-destructive examination images.

26. The method of claim 25 wherein the cine loop is rotated in an about 30-60 degree arc.

27. The method of claim 1 wherein the series of images comprises a laterally-moving series of images whereby a subject is sliced by the images.

28. The method of claims 1 or 26 wherein the series of images comprises a series of images recorded of substantially the same site and over time.

29. The method of claims 1 or 26 wherein the series of images comprises a video or movie image sequence.

30. The method of claim 1 wherein the method further comprises automating at least one image variable selected from the group consisting of Z-axis height, roll, contrast, center movement, and directional lighting.

31. The method of claim 30 wherein the image variable is directional lighting and the method further comprises holding the image stationary, and alternating the apparent lighting of an object in the image between point source lighting and directional lighting.

32. The method of claim 30 wherein the image variable is directional lighting and the apparent light source moves between different positions in the image.

33. The method of claim 30 wherein the image variable is apparent motion within the image and the center of an object in the image appears to move closer to the screen, then recedes from it.

34. The method of claim 1 wherein the method further comprises providing at least one of a sound indicator or an optical indicator that indicates the pause occurred.

35. Computer-implemented programming that performs the automated elements of the method of claim 1.

36. A computer comprising computer-implemented programming that performs the automated elements of the method claim 1.

37. The computer of claim 36 wherein the computer comprises a distributed network of linked computers.

38. The computer of claim 36 wherein the computer comprises a handheld computer, and the method of claim 1 is implemented on the handheld computer.

39. The computer of claim 36 wherein the computer comprises a wirelessly connected computer, and the method claims 1 is implemented on the wireless computer.

40. A networked computer system comprising computer-implemented programming that performs the automated elements of the method of claim 1.

41. The networked computer system of claim 40 wherein the networked computer system comprises a handheld wireless computer, and the method of claim 1 is implemented on the handheld wireless computer.

42. A networked computer system comprising a computer according to claim 36.
